(19) United States
(12) Patent Application Publication
(10) Pub. No.: US 2025/0325783 A1
(43) Pub. Date: Oct. 23, 2025

(54) TEFLON LINED BRAID WIRE CATHETER FOR ENHANCED TRACKABILITY

(71) Applicant: DEPUY SYNTHES PRODUCTS, INC., RAYNHAM, MA (US)

(72) Inventors: PEDRO D. PEDROSO, PARKLAND, FL (US); CHADWIN HANNA, JR., RAYNHAM, MA (US)

(73) Assignee: DEPUY SYNTHES PRODUCTS, INC., RAYNHAM, MA (US)

(21) Appl. No.: 18/641,641

(22) Filed: Apr. 22, 2024

(51) Int. Cl. A61M 25/00

(52) U.S. Cl. CPC A61M 25/0045; A61M 25/005

(57) ABSTRACT

A catheter that includes an inner lumen liner comprising a first polymer material, a braided metal wire jacket comprising a plurality of metal wires coated in the first polymer material, and an outer liner comprising a second polymer material different from the first polymer material. A portion of the plurality of metal wires are partially exposed on an outer surface of the outer liner and the coating on the exposed portion of the plurality of metal wires forms a contact surface.

### FIELD

The present invention generally relates to medical instruments, and more particularly, to embolic implants for aneurysm therapy.

### BACKGROUND

Several types of catheters are used during neurovascular interventions. Regardless of the type of catheter being utilized, each catheter will need to be able to perform three general functions: track through a specific anatomy while achieving the lowest friction possible, reach a treatment site as efficiently as possible, and (depending on the type of catheter being utilized) accept an implant, catheter, or other device within its lumen. In order for catheters to reduce track forces, the catheter needs to be able to achieve as little friction as possible as it navigates through the vasculature of the patient. This can be particularly challenging due to the nature of the vasculature in which the catheter is navigating.

Current methods for achieving as little friction as possible and reducing the track forces include applying lubricious coating to the outer surfaces of the catheters. Unfortunately, these coatings have poor integrity and can rub off along the vasculature as the catheters navigate through. These coatings can also be expensive to employ.

As a result, there remains a need for improved catheter designs that refine current methods for reducing the track forces that the catheter experiences. The presently disclosed designs are aimed at providing an improved catheter layering of polymer materials and a braided metal wire jacket capable of reducing friction and methods for fabricating such a catheter.

### SUMMARY

In some examples, a catheter is disclosed for treating aneurysms. The catheter can include a central polymer liner comprising a first polymer material, a braided metal wire jacket including a plurality of metal wires having at least one metal wire coated in the first polymer material. In some examples, a portion of the plurality of metal wires can be partially exposed on an outer surface of the central polymer liner and a portion of the plurality of metal wires can be partially exposed on an inner surface of the central polymer liner. In some examples, the coating on the exposed portions of the plurality of metal wires can form contact surfaces.

In some examples, the partially exposed outer surface and the partially exposed inner surface can be chemically etched.

In some examples, the partially exposed outer surface of the plurality of metal wires can be the contact surface with the vessel wall.

In some examples, every other metal wire of the plurality of metal wires can be coated in the first polymer material.

In some examples, only a portion of the plurality of metal wires can be coated in the first polymer material such that a spiral pattern is created along the catheter.

In some examples, the catheter can include an inner polymer liner, comprising a second polymer material different from the first polymer material, the inner polymer liner disposed over the partially exposed inner surface of the plurality of metal wires.

In some examples, an outer surface of the inner polymer liner and the partially exposed inner surface of the plurality of metal wire can be chemically etched.

In some examples, the outer surface of the inner polymer liner can form a contact surface with the partially exposed inner surface of the plurality of metal wires and the partially exposed outer surface of the plurality of metal wires can form a contact surface with the vessel wall.

In some examples, the plurality of metal wires can include a first layer of metal wires having a first diameter alternating with a second layer of metal wires having a second diameter, and such that the first diameter of the first layer of metal wires is greater than the second diameter of the second layer of metal wires.

In some examples, the catheter can include an outer polymer liner, comprising a third polymer material different from the first and second polymer materials, the outer polymer liner disposed over the partially exposed outer surface of the plurality of metal wires.

In some examples, an inner surface of the outer polymer liner and the partially exposed outer surface of the plurality of metal wires can be chemically etched.

In some examples, the partially exposed outer surface can form a contact surface with the inner surface outer polymer liner and an outer surface of the outer polymer liner can form a contact surface with the vessel wall.

In some examples, the plurality of metal wires can include a first layer of metal wires having a first diameter alternating with a second layer of metal wires having a second diameter, and such that the first diameter of the first layer of metal wires is greater than the second diameter of the second layer of metal wires.

In some examples, the catheter can include an inner polymer liner disposed over the partially exposed inner surface of the plurality of metal wires and an outer polymer liner disposed over the partially exposed outer surface of the plurality of metal wires.

In some examples, a method of manufacturing a catheter is disclosed. The method can include braiding a metal wire jacket comprising a first liner of metal wires pre-coated with a polymer material, and a second, alternating liner of metal wires, over a mandrel; re-flowing a central polymer liner comprising the same polymer material over the braided metal wire jacket; partially exposing an outer surface of the plurality of metal wires on an outer surface of the central polymer liner; partially exposing an inner surface of the plurality of metal wires on an inner surface of the central polymer liner; and removing the mandrel.

In some examples, the method can further include chemically etching the outer surface and the inner surface of the plurality of metal wires of the braided metal wire jacket.

In some examples, the method can further include, before braiding the metal wire jacket, placing an inner polymer liner comprising a second polymer material over the mandrel such that an inner surface of the plurality of metal wires forms a contact surface with the inner polymer liner.

In some examples, the method can further include the step of, before removing the mandrel, re-flowing an outer polymer liner comprising a third polymer material over the partially exposed outer surface of the plurality of metal wires such that the partially exposed outer surface forms a contact surface with the outer polymer liner and an outer surface of the outer polymer liner is the contact surface with the vessel wall.

In some examples, the step of braiding a metal wire jacket pre-coated with the first polymer material can include braiding two layers of metal wires wherein a first layer of metal wires comprises a first diameter and a second layer of metal wires comprises a second diameter such that the first diameter of the first layer of metal wires is greater than the second diameter of the second layer of metal wires.

In some examples, the first polymer material of the central polymer liner can comprise Teflon and the second polymer material of the inner polymer liner and the third polymer material of the outer polymer layer can comprise Pebax.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is a view of a blood vessel and a clot and an example catheter according to aspects of the present invention.

FIG. 2 is a side view of a funnel-mouth catheter having a braided metal wire jacket according to aspects of the present invention.

FIG. 3A illustrates a cross-section of the catheter in FIG. 2 according to aspects of the present invention.

FIG. 3B illustrates a magnified section of the cross-section of FIG. 3A detailing the coating of the plurality of metal wires of the braided metal wire jacket according to aspects of the present invention.

FIG. 3C illustrates an example embodiment of the catheter with the exposed portion of the plurality of metal wires showing according to aspects of the present invention.

FIG. 4A illustrates an alternate cross-section of the catheter in FIG. 2 having an additional layer of polymer material according to aspects of the present invention.

FIG. 4B illustrates a magnified section of the cross-section of FIG. 4A detailing the coating of the plurality of metal wires of the braided metal wire jacket and the additional layer of polymer material according to aspects of the present invention.

FIG. 5A illustrates an alternate cross-section of the catheter in FIG. 2 having both portions of the plurality of metal wires exposed.

FIG. 5B illustrates a magnified section of the cross-section of FIG. 5A detailing the coating of the plurality of metal wires of the braided metal wire jacket.

FIG. 6 is a flow-diagram illustrating a method of manufacture for a catheter according to aspects of the present invention.

FIGS. 7A-7D illustrate a method of manufacture for a catheter according to aspects of the present invention.

### DETAILED DESCRIPTION

The following detailed description is merely exemplary in nature and is not intended to limit the disclosure or the application and uses of the disclosure. Although the description of the disclosure is in many cases in the context of treatment of intracranial arteries, the disclosure may also be used in other body passageways as previously described.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human e.g., rat, dog, pig, monkey, or the like.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a,” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

Accessing cerebral, coronary and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this disclosure and do not need to be described in detail.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

A common theme across many of the disclosed designs is a catheter that includes an inner lumen liner comprising a first polymer material, a braided metal wire jacket comprising a plurality of metal wires coated in the first polymer material, and an outer liner comprising a second polymer material different from the first polymer material. A portion of the plurality of metal wires are partially exposed on an outer surface of the outer liner and the coating on the exposed portion of the plurality of metal wires forms a contact surface.

The braided metal wire jacket of the designs disclosed are desirably made from a material capable of recovering its shape automatically once released from a highly strained delivery configuration. A superelastic material such as Nitinol or an alloy of similar properties is particularly suitable. The material can be in many forms such as wire or strip or sheet or tube. A particularly suitable manufacturing process is to laser cut a Nitinol tube and then heat set and electropolish the resultant structure to create a framework of struts and connecting elements. This framework can be any of a huge range of shapes as disclosed herein and may be rendered visible under fluoroscopy through the addition of alloying elements such as platinum or through a variety of other coatings or marker bands.

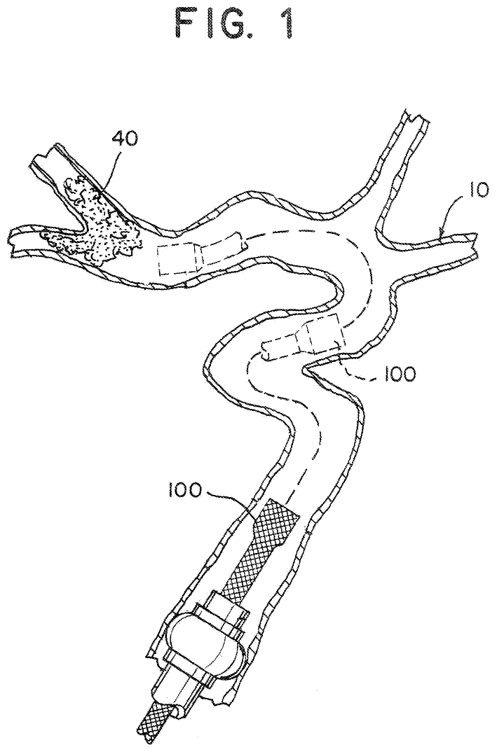

FIG. 1 is an illustration of a possible sequence for approaching an occlusive clot 40 within a vascular system 10 using a catheter 100 of the designs disclosed herein. The catheter 100 can be highly flexible such that it is capable of navigating vascular system 10 or other tortuous regions of the neurovascular system to reach an occlusive clot.

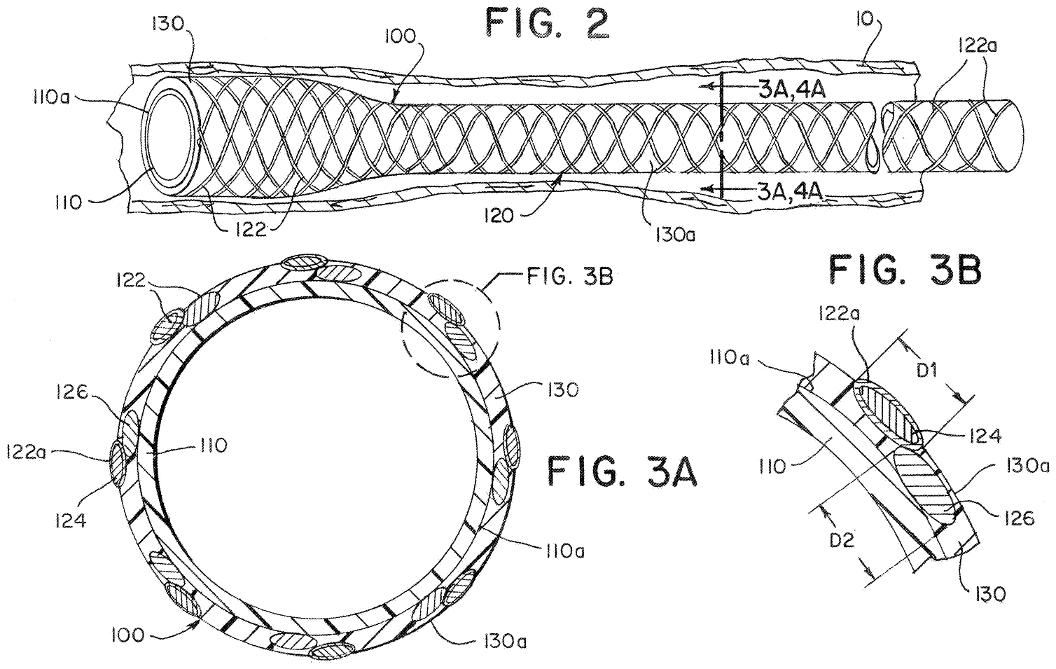

FIG. 2 is a side view of an exemplary funnel-mouth catheter 100 within a vascular system 10.

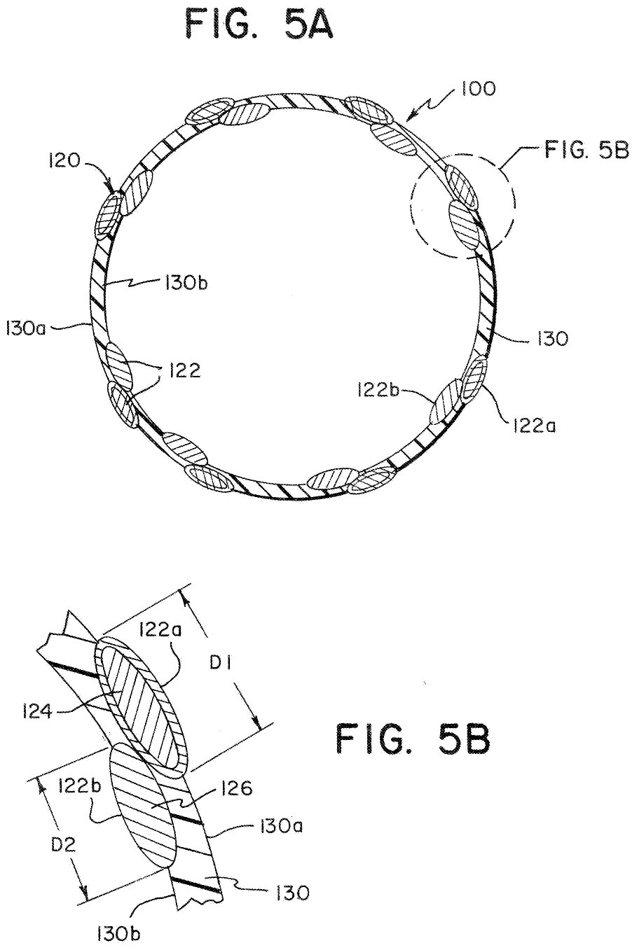

In some examples, the catheter 100 can include a central polymer liner 130, a braided metal wire jacket 120 comprising a plurality of metal wires 122, and an inner polymer liner 110. In some examples, a portion 122a of the plurality of metal wires 122 can be partially exposed on an outer surface 130a of the central polymer liner 130. In some examples, as will be shown in further detail in FIGS. 5A-5B, a portion 122b of the plurality of metal wires 122 can be partially exposed on an inner surface 130b of the central polymer liner 130 and form a contact surface with the inner polymer liner 110. In some examples, the coating on the exposed portion 122a of the plurality of metal wires can form a contact surface either with the vasculature 10 or another liner, both described in detail below.

In some examples, an outer surface 110a of the inner lumen liner 110 and the partially exposed outer surface 122a of the plurality of metal wires can be chemically etched.

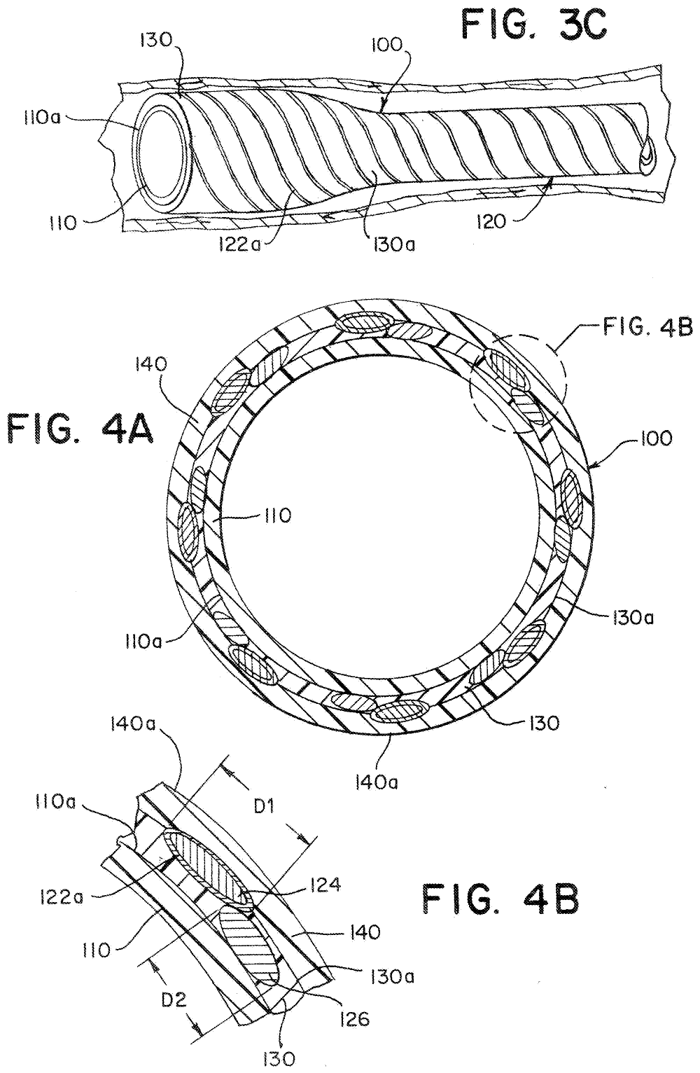

FIG. 3A illustrates a cross-section of the catheter 100 in FIG. 2, detailing the layers of polymer materials and the braided metal wire jacket. In some examples, the central polymer liner 130 and the plurality of metal wires 122 of the braided metal wire jacket 120 partially exposed 122a on the outer surface 130a of the central polymer liner 130 can include a first polymer material. The inner polymer liner 110 can include a second polymer material different from the first polymer material.

FIG. 3B illustrates a section of the cross-section of FIG. 3A detailing the coating of the plurality of metal wires 122 of the braided metal wire jacket 120. In some examples, the plurality of metal wires 122 can include a first layer of metal wires 124 and a second, alternating layer of metal wires 126. The coating on the exposed portion 122a of the plurality of metal wires can be applied to only the first layer of metal wires 124 and the exposed portion 122a can form a contact surface with the vessel wall 10.

Due to the coating on the plurality of metal wires 122, the metal wires 122 are able to adhere to the central layer 130, or central polymer liner, of polymer material efficiently. The exposed portion 122a of the plurality of metal wires 122 can have a lubricious coating, on at least the exposed side 122a, to facilitate smooth contact with the vessel wall 10. The coated exposed portion 122a also allows the catheter 100 to have an outer surface 130a coated in a first polymer material that does not require lubricious coating in order to navigate the vasculature 10. Because the wires 122 are pre-coated in a central layer 130 of polymer material, the polymer material provides a barrier around the outer lumen of the catheter 100 that is less likely to be removed during navigation unlike current methods involving a lubricious coating to the outer surface of a catheter.

FIG. 3C illustrates the exposed portion 122a of the plurality of metal wires 122 partially exposed on the outer surface 130a of the central polymer liner 130. In this embodiment, only the first layer 124 of the plurality of metal wires 122 is coated in the first polymer material and exposed on the outer surface 130a of the central polymer liner 130.

FIG. 4A illustrates an alternate cross-section of the catheter 100 in FIG. 2, detailing the layers of polymer materials and the braided metal wire jacket. In some examples, the central polymer liner 130 and the plurality of metal wires 122 of the braided metal wire jacket 120 partially exposed on the outer surface 130a of the central polymer liner 130 can include a first polymer material. The inner polymer liner 110 can include a second polymer material different from the first polymer material.

In some examples, the catheter 100 can further include an outer polymer liner 140 disposed on the central polymer liner 130 and the outer polymer liner 140 can include a third polymer material different from the first polymer material and the second polymer material. The coating on the exposed portion 122a of the plurality of metal wires 122 can be a contact surface with the outer polymer liner 140 and the outer polymer liner 140 can be a second contact surface 140a with the vessel wall 10.

FIG. 4B illustrates a section of the cross-section of FIG. 4A detailing the coating of the plurality of metal wires 122 of the braided metal wire jacket 120 and the outer polymer liner 140 of polymer material. In some examples, the plurality of metal wires 122 can include a first layer of metal wires 124 and a second, alternating layer of metal wires 126. The first layer of metal wires 124 can include a first diameter D1 and the second layer of metal wires 126 can include a second diameter D2 such that the first diameter D1 of the first layer of metal wires 124 is greater than the second diameter D2 of the second layer of metal wires 126.

The outer polymer liner 140 provides the benefit of adhering to the central layer 130 of polymer material through covalent bonding as well as adhering mechanically to the exposed portion 122a of the plurality of metal wires 122. This provides effective contact surfaces 122a, 130a between the central layer 130 and the outer layer 140 as well as a contact surface 140a between the outer layer 140 and the vessel wall 10. The outer layer 140 can have lubricious coatings to allow the catheter 100 to navigate the vasculature 10 efficiently. The different diameters D1, D2 also provide aid in regard to the wall strength of the catheter as well as a radially inhomogeneous construction to aid with adhesion to the outer layer 140 of polymer material. In other examples, the second layer of metal wires 126 having the second (smaller) diameter D2 can be the exposed wires forming the contact surface as discussed herein and the first layer of metal wires 124, having the first (larger) diameter D1 can be fully embedded in the second layer 130. Additional examples can have uniform diameter wires or multiple strands of wire in a layer or multiple layers. Other examples can include both the first and second layers 124, 126 providing a contact surface, either uniformly or can be woven in such a way to alternate exposure of the first and second layers.

FIG. 5A illustrates an alternate cross-section of the catheter 100 in FIG. 2. In this embodiment, an inner surface 122b and an outer surface 122a of the plurality of metal wires 122 are partially exposed on an inner surface 130b and an outer surface 130a of a central polymer liner 130, respectively. In some examples, the central polymer liner 130 and the plurality of metal wires 122 of the braided metal wire jacket 120 partially exposed 122a, 122b can include a first polymer material.

FIG. 5B illustrates a section of the cross-section of FIG. 5A detailing the coating of the plurality of metal wires 122 of the braided metal wire jacket 120. In some examples, the plurality of metal wires 122 can include a first layer of metal wires 124 and a second, alternating layer of metal wires 126. The coating on the exposed portions 122a, 122b of the plurality of metal wires can be applied to only the first layer of metal wires 124 and the outer exposed portion 122a can form a contact surface with the vessel wall 10.

Due to the coating on the plurality of metal wires 122, the metal wires 122 are able to adhere to the central layer 130, or central polymer liner, of polymer material efficiently. The exposed portions 122a, 122b of the plurality of metal wires 122 can have a lubricious coating, on at least the outer exposed side 122a, to facilitate smooth contact with the vessel wall 10. The coated exposed portion 122a also allows the catheter 100 to have an outer surface 130a coated in a first polymer material that does not require lubricious coating in order to navigate the vasculature 10. Because the wires 122 are pre-coated in a central layer 130 of polymer material, the polymer material provides a barrier around the outer lumen of the catheter 100 that is less likely to be removed during navigation unlike current methods involving a lubricious coating to the outer surface of a catheter.

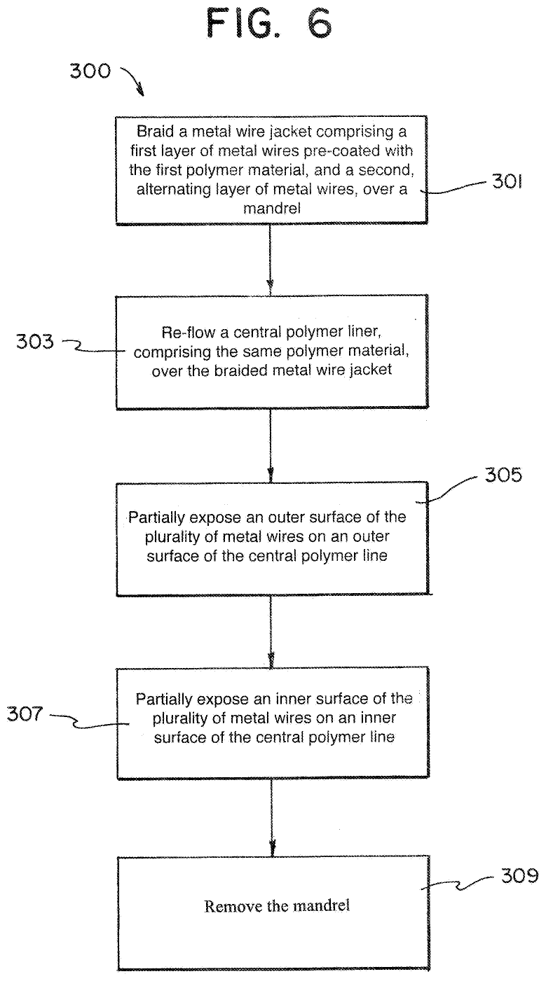

FIG. 6 shows a method 300 of manufacture for catheter 100 as disclosed herein. The method steps in FIG. 6 can be implemented by any of the example means described herein or by similar means, as will be appreciated.

At block 301, the method 300 can include braiding a metal wire jacket comprising a first liner of metal wires 124 pre-coated with a polymer material, and a second, alternating layer of metal wires 126, over a mandrel 50. The mandrel can be configured similarly to an example mandrel 50 disclosed herein, variations thereof, and alternatives thereto as understood by a person skilled in the pertinent art. The braided metal wire jacket can be configured similarly to an example braided metal wire jacket 120 disclosed herein, variations thereof, and alternatives thereto as understood by a person skilled in the pertinent art.

In some examples, the step of braiding a metal wire jacket 120 pre-coated with the first polymer material can comprise of only pre-coating and chemically etching every other metal wire 124 of the braided metal wire jacket 120 with the second polymer material.

In some examples, the step of braiding a metal wire jacket 120 pre-coated with the first polymer material can comprise of braiding two layers of metal wires 122 wherein a first layer of metal wires 124 comprises a first diameter D1 and a second layer of metal wires 126 comprises a second diameter D2 such that the first diameter D1 of the first layer of metal wires 124 is greater than the second diameter D2 of the second layer of metal wires 126.

In some examples, the method 300 can further include: before braiding the metal wire jacket 120, placing an inner polymer liner 110 comprising a second polymer material over the mandrel 50 such that an inner surface 122b of the plurality of metal wires 122 forms a contact surface with the inner polymer liner 110.

At block 303, the method 300 can include re-flowing a central polymer liner 130 comprising the polymer material over the braided metal wire jacket 120.

At block 305, the method 300 can include partially exposing an outer surface 122a of the plurality of metal wires on an outer surface 130a of the central polymer liner 130.

At block 307, the method 300 can include partially exposing an inner surface 122b of the plurality of metal wires 122 on an inner surface 130b of the braided metal wire jacket 120.

At block 309, the method 300 can include removing the mandrel 50.

In some examples, the method 300 can further include: before removing the mandrel 50, re-flowing an outer polymer liner 140 comprising a third polymer material over the partially exposed outer surface 122a of the plurality of metal wires 122 such that the partially exposed outer surface 122a forms a contact surface with the outer polymer liner 140 and an outer surface 140a of the outer polymer liner 140 is the contact surface with the vessel wall 10.

In some examples, the method 300 can further include chemically etching the outer surface 122a and the inner surface 122b of the plurality of metal wires 122 of the braided metal wire jacket 120.

In some examples, the first polymer material of the central polymer liner 130 can comprise Teflon and the second polymer material of the inner polymer liner 130 and the third polymer material of the outer polymer liner 140 can comprise Pebax.

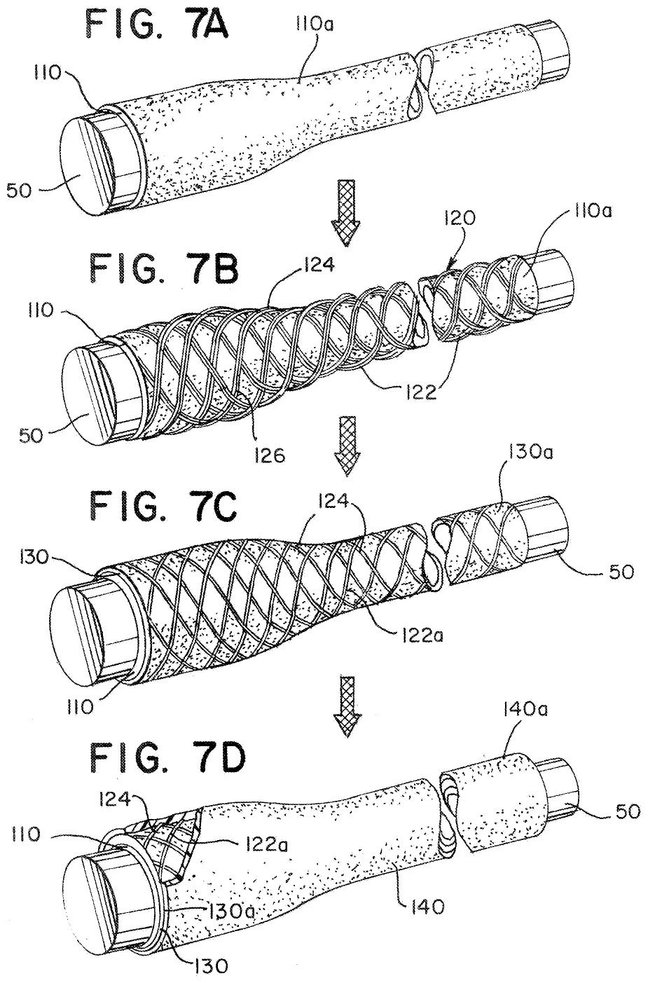

FIGS. 7A-D illustrate the method 300 of manufacture disclosed herein.

FIG. 7A illustrates the step in which the inner polymer liner 110 comprising a first polymer material is placed over a mandrel 50. The exposed outer surface 110a of the inner polymer liner 110 can be seen.

FIG. 7B illustrates the step in which a plurality of metal wires 122 is braided into a braided metal wire jacket 120 over the outer surface 110a of the inner polymer liner 110. The plurality of metal wires 122 can include a first layer of metal wires 124 and a second layer of metal wires 126.

FIG. 7C illustrates the step in which a central polymer liner 130 comprising a second polymer material over the braided metal wire jacket 120.

FIG. 7D illustrates the step of partially exposing an outer surface 122a of the plurality of metal wires 122 of the braided metal wire jacket 120. Also depicted here is an outer polymer liner 140 comprising a third polymer material different from the first and second polymer materials that has been re-flowed over the central polymer liner 130 such that an outer surface 140a of the outer polymer liner 140 is the contact surface for the vessel wall 10.

The descriptions contained herein are examples of embodiments of the invention and are not intended in any way to limit the scope of the invention. As described herein, the invention contemplates many variations and modifications of braided wire catheters, including inner diameter exposure, outer diameter exposure, both inner and outer diameter exposure, etc. Modifications and variations apparent to those having skilled in the pertinent art according to the teachings of this disclosure are intended to be within the scope of the claims which follow.

What is claimed is:

1. A catheter comprising:
a central polymer liner comprising a first polymer material; and
a braided metal wire jacket comprising a plurality of metal wires having at least one metal wire coated in the polymer material;
wherein a portion of the plurality of metal wires are partially exposed on an outer surface of the central polymer liner, and a portion of the plurality of metal wires are partially exposed on an inner surface of the central polymer liner; and wherein the coating on the exposed portions of the plurality of metal wires form contact surfaces.

2. The catheter of claim 1, wherein the partially exposed outer surface and the partially exposed inner surface of the plurality of metal wires is chemically etched.

3. The catheter of claim 1, wherein the partially exposed outer surface of the plurality of metal wires is the contact surface with the vessel wall.

4. The catheter of claim 1, wherein every other metal wire of the plurality of metal wires is coated in the first polymer material.

5. The catheter of claim 1, wherein only a portion of the plurality of metal wires is coated in the polymer material such that a spiral pattern is created along the catheter.

6. The catheter of claim 1, wherein the catheter comprises an inner polymer liner, comprising a second polymer material different from the first polymer material, the inner polymer liner disposed over the partially exposed inner surface of the plurality of metal wires.

7. The catheter of claim 6, wherein an outer surface of the inner polymer liner and the partially exposed inner surface of the plurality of metal wires are chemically etched.

8. The catheter of claim 7, wherein the outer surface of the inner polymer liner forms a contact surface with the partially exposed inner surface of the plurality of metal wires and the partially exposed outer surface of the plurality of metal wires forms a contact surface with the vessel wall.

9. The catheter of claim 6, wherein the plurality of metal wires comprises a first layer of metal wires having a first diameter alternating with a second layer of metal wires having a second diameter, and such that the first diameter of the first layer of metal wires is greater than the second diameter of the second layer of metal wires.

10. The catheter of claim 1, wherein the catheter comprises an outer polymer liner, comprising a third polymer material different from the first and second polymer materials, the outer polymer liner disposed over the partially exposed outer surface of the plurality of metal wires.

11. The catheter of claim 10, wherein an inner surface of the outer polymer liner and the partially exposed outer surface of the plurality of metal wires are chemically etched.

12. The catheter of claim 11, wherein the partially exposed outer surface forms a contact surface with the inner surface outer polymer liner and an outer surface of the outer polymer liner forms a contact surface with the vessel wall.

13. The catheter of claim 10, wherein the plurality of metal wires comprises a first liner of metal wires having a first diameter alternating with a second liner of metal wires having a second diameter, and such that the first diameter of the first liner of metal wires is greater than the second diameter of the second liner of metal wires.

14. The catheter of claim 1, wherein the catheter comprises an inner polymer liner disposed over the partially exposed inner surface of the plurality of metal wires and an outer polymer liner disposed over the partially exposed outer surface of the plurality of metal wires.

15. A method of forming a catheter comprising:
braiding a metal wire jacket comprising a first layer of metal wires pre-coated with a polymer material, and a second, alternating layer of metal wires, over a mandrel;
re-flowing a central polymer liner, comprising the polymer material, over the braided metal wire jacket;
partially exposing an outer surface of the plurality of metal wires on an outer surface of the central polymer liner;
partially exposing an inner surface of the plurality of metal wires on an inner surface of the braided metal wire jacket; and
removing the mandrel.

16. The method of claim 15, further comprising:
chemically etching the outer surface and the inner surface of the plurality of metal wires of the braided metal wire jacket.

17. The method of claim 15, wherein the method comprises:
before the step of braiding the metal wire jacket, placing an inner polymer liner comprising a second polymer material over the mandrel such that an inner surface of the plurality of metal wires forms a contact surface with the inner polymer liner.

18. The method of claim 15, wherein the method comprises:
before the step of removing the mandrel, re-flowing an outer polymer liner comprising a third polymer material over the partially exposed outer surface of the plurality of metal wires such that the partially exposed outer surface forms a contact surface with the outer polymer liner and an outer surface of the outer polymer liner is the contact surface with the vessel wall.

19. The method of claim 15, wherein the step of braiding a metal wire jacket comprises braiding two liners of metal wires wherein a first liner of metal wires comprises a first diameter and a second liner of metal wires comprises a second diameter such that the first diameter of the first liner of metal wires is greater than the second diameter of the second liner of metal wires.

20. The method of claim 15, wherein the first polymer material of the central polymer liner comprises Teflon and wherein the second polymer material of the inner polymer liner and the third polymer material of the outer polymer liner comprises Pebax.
